(19) United States
(12) Patent Application Publication
(10) Pub. No.: US 2009/0143471 A1
(43) Pub. Date: Jun. 4, 2009

(54) Process for the Atomization of Ioxilan

(75) Inventors: Myriam Petta, Montmorency (FR); Damien Lavergne, Sceaux (FR)

Correspondence Address: BIRCH STEWART KOLASCH & BIRCH, PO BOX 747, FALLS CHURCH, VA, 22040-0747, US

(73) Assignee: GUERBET, Villepinte (FR)

(21) Appl. No.: 12/226,063

(22) PCT Filed: Apr. 6, 2007
(86) PCT No.: PCT/EP2007/053431

§ 371 Date: Oct. 7, 2008

Related U.S. Application Data
(60) Provisional application No. 60/795,202, filed on Apr. 27, 2006.

(30) Foreign Application Priority Data
Apr. 7, 2006 (FR) 0603088

(51) Int. Cl. A61K 31/166; C07C 233/69

(52) U.S. Cl. 514/616; 564/153

(57) ABSTRACT

The invention relates to a process for the preparation of ioxilan of formula 5-[N-(2,3-dihydroxypropyl)acetamido]-2,4,6-triiodo-N-(2,3-dihydroxypropyl)-N′-(2-hydroxyethyl)isophthalamide by atomization and to the improved product thus obtained. This process makes it possible to avoid a process by crystallization and results in an active principle having an improved solubility.

The invention relates to a process for the preparation of ioxilan, of formula 5-[N-(2,3-dihydroxypropyl)acetamido]-2,4,6-triiodo-N-(2,3-dihydroxypropyl)-N′-(2-hydroxyethyl)isophthalamide, and to the improved product thus obtained. Ioxilan is the active principle of a commercial nonionic contrast agent for X-ray imaging (Oxilan®, Imagenil®). The manufacture of ioxilan, disclosed in particular in U.S. Pat. No. 4,954,348, comprises the preparation of a purified active principle and then its pharmaceutical formulation, in order to obtain the iodinated contrast agent administered to man.

Various processes exist for the preparation of ioxilan. They make it possible to obtain crude ioxilan in solution, denoted A in the remainder of the patent application and of following general formula (I):

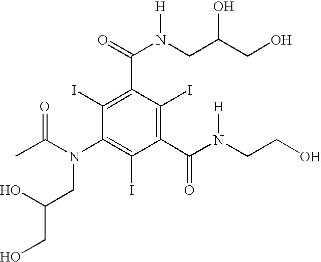

the compound A in solution being subsequently to be isolated to give a solid compound B.

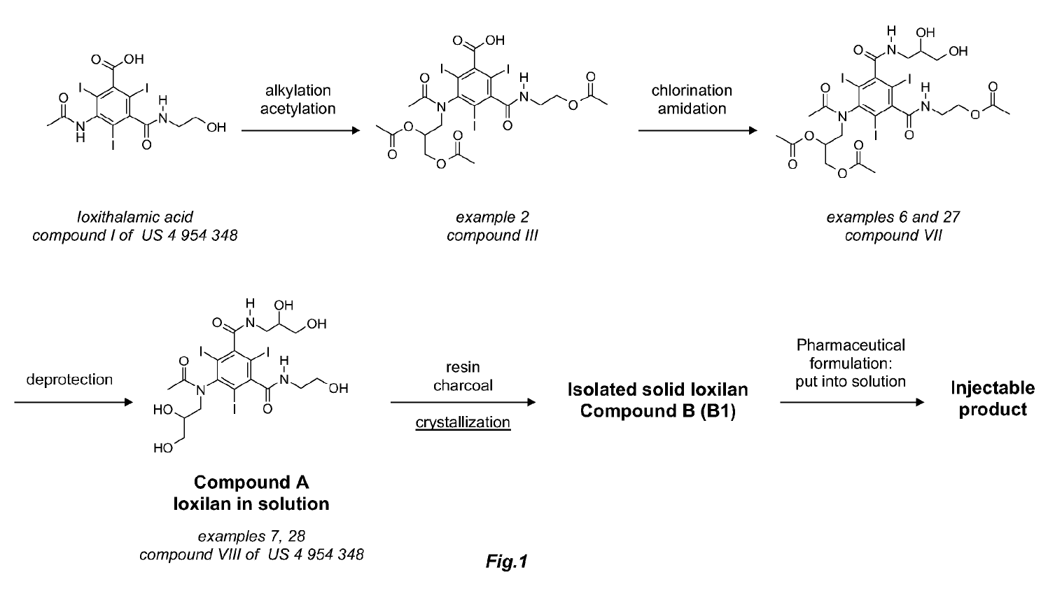

Thus, in order to obtain the compound A in solution, an appropriate process comprises the stages, restated in the appended FIG. 1, with the references and compound numbers taken from U.S. Pat. No. 4,954,348 (ioxilan in solution is described under the compound (VIII) reference of this document). In short, compound A is prepared from ioxithalamic acid according to the following successive stages: alkylation with chloropropanediol, acetylation with acetic anhydride, chlorination with thionyl chloride, amidation with aminopropanediol, deacetylation deprotection.

Product B of formula (I), before the pharmaceutical formulation stage, was isolated in the prior art by crystallization of the compound A solution from an alcoholic solvent, preferably methanol or ethanol (it being specified that the Applicant Company has also studied crystallization from other alcohols, such as propanol, butanol, isopropanol or methoxyethanol derivatives, in particular). If appropriate, the crystallization was preceded by a treatment over active charcoal and by a treatment over acidic or basic resins, to remove impurities (coloration, related organic impurities, ionic impurities) from the solution of compound A, followed by a drying stage, in order to remove possible solvent residues. This process by crystallization was expensive and complex, and generated a large amount of alcoholic effluents which it was necessary to destroy. These drawbacks became more disadvantageous as the amounts of active principle to be manufactured increased, due to the high volume of contrast agent injected.

After major studies, the Applicant Company has henceforth succeeded in obtaining the active principle ioxilan, isolated and of satisfactory quality, by replacing the crystallization stage by an atomization. To this end, the invention relates to a process for the preparation of the isolated ioxilan compound of formula (I),

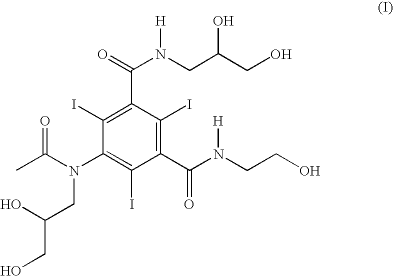

comprising the atomization of an aqueous solution of ioxilan of general formula (I). The invention thus relates to a process for the isolation of ioxilan of formula (I) comprising the atomization of an aqueous solution of ioxilan. Advantageously, the atomization is preceded by a treatment over charcoal, by a treatment over resins or by other analogous treatments, in order to remove impurities from the solution of crude compound A. It is thus possible to obtain the active principle ioxilan without using an alcoholic solvent in the final isolation stage, which represents a marked improvement from the viewpoint of simplicity and cost and a reduction in the impact on the environment (less in the way of organic effluents).

The invention also relates to a process for the preparation of a pharmaceutical ioxilan composition from the ioxilan isolated by atomization. This process comprises the aqueous dissolution of the atomized ioxilan and of appropriate pharmaceutically acceptable excipient(s).

Another disadvantage of the prior process by crystallization was that, in order to obtain an appropriate concentration of active principle in the contrast agent, the dissolution of the crystalline compound B1 (solid crystalline ioxilan, denoted B1; compound B of FIG. 1 is compound B1) required heating at high temperature (80° C.). This is because the immediate solubility of B1 in water at ambient or moderate temperature (typically from 20 to 40° C.) was of the order of 50 mg of iodine/ml.

In point of fact, surprisingly for a person skilled in the art, the active principle obtained by atomization (solid atomized ioxilan, denoted B2) exhibits physicochemical properties, in particular of immediate solubility in water, which are improved and unexpected in comparison with the active principle obtained by crystallization (solid crystalline ioxilan, denoted B1). The atomized compound B2 is soluble at ambient or moderate temperature. It is possible, starting from the compound B2, to obtain an appropriate pharmaceutical solution without needing to heat at high temperature. The immediate solubility of B2 in water at ambient or moderate temperature, of the order of at least 500 mg of iodine/ml and typically of approximately 500 mg of iodine/ml, makes it possible to obtain an injected contrast agent having a concentration of 350 to 450 mg of iodine/ml, for example 350, 380 or 400 mg of iodine/ml.

This substantial improvement in the immediate solubility is very useful in facilitating and simplifying the manufacture of the pharmaceutical solution and in particular the initial stage of dissolution of the active principle.

The product obtained retains its character of nonionic monomeric product of low osmolality.

As the active principle B2 is a novel compound with respect to the active principle B1, the invention relates, according to another aspect, to the active principle B2 capable of being obtained by a process for the preparation of the compound ioxilan isolated by atomization (the said process comprising the preparation of an aqueous solution of a compound of formula (I) and then the atomization of this solution).

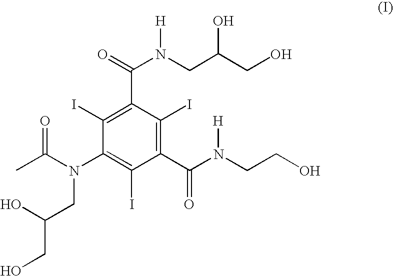

In order to explain this different a posteriori, a difference in the isomeric profile can be assumed between the compounds B1 and B2, the compound B2 exhibiting an isomeric profile advantageous for the solubility between endo isomers and exo isomers of ioxilan (orientation of the C═O group towards or away from the ring). The atomization process can use highly varied techniques and items of equipment. Advantageously, for the compound ioxilan, the atomization device is adjusted in order to have an outlet temperature of the device of less than 130° C., in order not to risk decomposing the product. The feed flow rate and the inlet temperature are appropriately adjusted according to the power of the device, in order not to exceed 130° C. (preferably not to exceed 110° C.) at the outlet, the drying rate consequently being higher or lower. The concentration of the solution A to be atomized and the drying rate are defined according to the device, with typically a concentration of 5 to 385 mg of iodine/ml, for example from 215 to 265 mg of iodine/ml, with a temperature of between 10 and 60° C., it being known that a dilute solution typically results in a reduction in the size of the particles of the powder (B2) obtained. The size can be varied, from the order of a micron to several tens or hundreds of microns.

By way of nonlimiting example, highly satisfactory results in terms of control of the atomization process and of product appearance (fine powder) are obtained using a co- or countercurrent atomizer with nozzle or rotating disc. The gas used is typically air or an inert gas. The temperature of the gas at the outlet is such that the ioxilan is not decomposed. The temperature at the inlet is, for example, between 130 and 250° C. and, at the outlet, less than 130° C., typically from 100 to 110° C. It is possible, for example, to use a pneumatic nozzle of 0.5 to 1 mm. The flow rate of the solution is, for example, from 1 to 100 ml/min, in particular from 20 to 40 ml/min. The temperature of the solution is, for example, from 10 to 60° C.

More generally, the term “atomization process” is understood to mean, within the meaning of the present invention, any drying process which is appropriate so as to obtain an ioxilan powder, which includes, for example, dehydration processes, spray drying or drying using a disperser.

Advantageously, the aqueous ioxilan solution results from a process for the manufacture of ioxilan without a crystallization stage.

The pharmaceutical process for the preparation of the contrast agent comprises the aqueous dissolution of the compound B2 using appropriate excipients, the solution subsequently being sterilized (typically at 121° C.) and stored in a sterile packaging (bottle, bag), it being possible for the storage time to be several months before opening. By observing the appropriate conditions for storage and use, the concentration of the injected solution is typically from 300 to 500 mg of iodine/ml of solution, advantageously from 350 to 450 mg of iodine/ml.

The invention also relates to a process for the preparation of a pharmaceutical ioxilan composition (for diagnosis) comprising the aqueous dissolution of ioxilan isolated by atomization B2 of formula (I) and of appropriate pharmaceutically acceptable excipients. The invention also relates to a solution, advantageously an injectable solution, comprising ioxilan B2 obtained by atomization and having a concentration of 350 to 450 mg of iodine/ml.

The compound ioxilan thus obtained and placed in pharmaceutical solution so as to obtain an iodinated contrast agent is intended in particular for the following indications: coronary, peripheral or cerebral angiography or arteriography, left ventriculography, or any other known indication of iodinated products of low osmolality, according to the appropriate methods of administration known to a person skilled in the art. It is also possible to combine ioxilan with other contrast agents, in particular nonionic monomers or dimers. Mention will be made, among formulating agents, of the following organic or inorganic pharmaceutically acceptable excipients (for diagnosis): water, saline solution, stabilizing agents, such as calcium or sodium EDTA, sodium chloride, trometamol, buffers, or any other additive mentioned in particular in U.S. Pat. No. 4,954,348 or in any patent or publication on nonionic monomers. Mention will be made, for example, of formulations based on: sodium, sodium/calcium mixture (U.S. Pat. No. 5,328,680), TRIS (U.S. Pat. No. 5,349,085), organic amine and carboxylic acid mixture (US2005/00025711), various additives of U.S. Pat. No. 5,695,742 (anticoagulants, and the like). For example, the formulation will be, for 1 ml of injectable solution:
ioxilan:
   623.4 mg/ml for a 300 mg of iodine/ml pharmaceutical solution,
   727.3 mg/ml for a 350 mg of iodine/ml pharmaceutical solution,
   831.2 mg/ml for a 400 mg of iodine/ml solution,
edetate calcium disodium (complexing agent): 0.561 mg/ml
buffer: anhydrous citric acid (0.576 mg/ml)
sodium hydroxide and carbon dioxide for adjusting the pH to 5.5-7,5
water (solvent): volume for making up to 1 ml.

The detailed description which follows describes the preparation of compound A.

### SYNTHESIS OF COMPOUND A

Compound A in solution is prepared as in Example 7 (compound VIII) of U.S. Pat. No. 4,954,348, except that, at the end of this example, the aqueous solution of the product is atomized using an atomization drying device in order to result in an isolated solid B2 in the form of a fine white powder.

### Stage 1. Alkylation of Ioxithalamic Acid

### 5-acetamido-2,4,6-triiodo-N-(2-hydroxyethyl)isophthalamic Acid to Give

### 5-[N-(2,3-dihydroxypropyl)acetamido]-2,4,6-triiodo-N-(2-hydroxyethyl)-isophthalamic Acid

A 1N aqueous sodium hydroxide solution (250 ml) was added to ioxithalamic acid (161 g, 0.25 mol) and the pH was adjusted with 5N NaOH to 10.5-10.6 at 85-90° C. 3-Chloro-1,2-propanediol (30.41 g, 0.275 mol) was added and the pH was readjusted to between 10.5 and 10.6 with 5N NaOH with subsequent further additions after 1 hour (2.76 g, 0.025 mol) and after 2 hours (2.76 g, 0.025 mol). The reaction was complete after 2 and a half hours (monitoring by TLC).

Glacial acetic acid (5 ml) was added to pH 5, the solvents were evaporated and the residue was subjected to azeotropic entrainment with toluene (150 ml) in order to obtain 294 g of a mixture which was used without isolation of the product in the following stage.

### Stage 2. Acetylation of the N-alkylated Ioxithalamic Acid

### 5-[N-(2,3-dihydroxypropyl)acetamido]-2,4,6-triiodo-N-(2-hydroxyethyl)-isophthalamic Acid to Give

### 5-[N-(2,3-diacetoxypropyl)acetamido]-2,4,6-triiodo-N-(2-acetoxyethyl)-isophthalamic Acid

The crude mixture (290 g) from stage 1, comprising the title compound (250 mmol), was suspended in acetic anhydride (500 ml) and pyridine (19.76 g, 250 mmol) and was maintained under mechanical stirring at 65° C. By TLC monitoring, the acetylation was complete after 3 hours.

The acetic anhydride and acetic acid were evaporated and the residue was subjected to azeotropic entrainment with toluene (100 ml×2). The residue was dissolved in a saturated aqueous sodium bicarbonate solution (500 ml) and ethyl acetate (200 ml). The layers were separated and the layer comprising the bicarbonate was reextracted with ethyl acetate (200 ml×2). The aqueous layer was acidified with concentrated hydrochloric acid to pH 0-1 to produce a white precipitate, which was extracted with ethyl acetate (3×200 ml). The organic extracts were combined, washed with sodium chloride solution (100 ml) and dried over MgSO₄. The removal of the solvent gave 206 g of the product in the form of a white foam (97% yield).

### Stage 3. Formation of the Acyl Chloride of N-alkylated, Acetylated, Ioxithalamic acid

### 5-[N-(2,3-diacetoxypropyl)acetamido]-2,4,6-triiodo-N-(2-acetoxyethyl)-isophthalamic Acid to Give

### 5-[N-(2,3-diacetoxypropyl)acetamido]-2,4,6-triiodo-N-(2-acetoxyethyl)-isophthalamoyl Chloride

The compound from the preceding stage (250 g, 243 mmol) was dissolved in thionyl chloride (400 ml) and the reaction mixture was heated at 60-65° C. for 1 hour until the end of the reaction (monitoring by TLC). The thionyl chloride was evaporated using a rotary evaporator, the residue was subjected to azeotropic entrainment with ethyl acetate (250 ml×2) and the product was dissolved in ethyl acetate (400 ml), extracted with a saturated aqueous bicarbonate solution (150 ml×2), dried over MgSO₄ and then evaporated to dryness to give 202 g of a virtually white foam (96% yield).

### Stage 4. Amidation of the Alkylated, Acetylated, Ioxithalamic Acid

### 5-[N-(2,3-diacetoxypropyl)acetamido]-2,4,6-triiodo-N-(2-acetoxyethyl)-isophthalamoyl chloride with 3-amino-1,2-propanediol to Give

### 5-[N-(2,3-diacetoxypropyl)acetamido]-2,4,6-triiodo-N-(2-acetoxyethyl)-N′-(2,3-dihydroxypropyl)isophthalamide

The compound from the preceding stage (86.25 g, 100 mmol) was dissolved in dimethylacetamide (200 ml), where triethylamine (13.9 g, 100 mmol) and 3-amino-1,2-propanediol (10.93 g, 120 mmol) were added. The reaction mixture was stirred at ambient temperature for 8 hours until the reaction was at an end by TLC monitoring. The solvent was evaporated under vacuum and the product was dissolved in tetrahydrofuran (75 ml) to which sodium chloride solution had been added. The organic extract was washed with a sodium chloride solution/1N hydrochloric acid mixture (9:1, 50 ml×2), subsequently with a sodium chloride solution/water (1:1) mixture (50 ml×2) and finally with sodium chloride solution (40 ml×1). The organic layer was dried over MgSO₄ and the solvent removed to give 80.6 g of the product in the form of a virtually white foam (87.9% yield).

### Stage 5. Deprotection of the Alkylated, Acetylated, Amidated with 3-amino-1,2-propanediol, Ioxithalamic Acid

### 5-[N-(2,3-diacetoxypropyl)acetamido]-2,4,6-triiodo-N-(2-acetoxyethyl)-N′-(2,3-dihydroxypropyl)isophthalamide to Give

### 5-[N-(2,3-dihydroxypropyl)acetamido]-2,4,6-triiodo-N-(2,3-dihydroxypropyl)-N′-(2-hydroxyethyl)isophthalamide

The compound from the preceding stage (45.85 g, 50 mmol) was placed in methanol (100 ml) and a 1M solution of sodium methoxide in methanol (10.5 ml) was added. The solution was stirred for 30 minutes in order to obtain complete deprotection (by TLC and HPLC monitoring) and, during this time, methyl acetate was removed by vacuum distillation of its azeotrope with methanol. The solution was neutralized to pH 7 with Dowex 50 H+ resin and diluted with water (75 ml), and the methanol was removed by vacuum distillation. The compound VIII of U.S. Pat. No. 4,954,348 was thus obtained in aqueous solution, also denoted compound A of formula (I). Compound A in solution was decoloured with active charcoal.

Unlike the prior art, instead of being crystallized from a solvent (see column 12, line 38, of the document U.S. Pat. No. 4,954,348), the aqueous ioxilan solution obtained by the Applicant Company was atomized using an atomization drying device to result in a solid in the form of a fine white powder of isolated compound B2 (33.5 g; purity: 98.5-99.5%; 85% yield).

Parameters for adjusting the Buchi B290 atomization device: inlet air temperature: 210-220° C., outlet air temperature: 100-105° C.; feed flow rate of the solution: 33 ml/min; suction power of the fan: 100%; feed flow rate of air to the nozzle: 400 l/h.

NMR: (¹H, 80 MHz, d₆-DMSO): 8.6 (2H, broad multiplet, carbamoyl N—H); 4.9-4.0 (5H, broad singlet, exchangeable, hydroxyl protons); 4.1-2.8 (14H, multiplet, protons on carbon carrying nitrogen and hydroxyl functional groups); 2.25 and 1.8 (3H, pair of singlets, acetanilide methyl protons).

TLC: silica gel; 70:30 CHCl₃/MeOH: Rf (acetylated compound VII) 0.84; Rf (compound VIII produced) 0.20.

HPLC: aminopropyl Alltech, 10μ, 31 ml/min 87% acetonitrile/water, Rf: 6.1 and 7.5 for two isomers.

The preparative route described above for a solution of compound A is not limiting. In addition to the other synthetic routes disclosed in U.S. Pat. No. 4,954,348, it is also possible to use, for example, a synthesis according to the following successive stages: acetylation (with acetic anhydride and an optional catalyst), chlorination (with thionyl chloride and an optional catalyst), amidation (with addition of triethylamine and then of aminopropanediol), deacetylation/alkylation (with addition of sodium hydroxide and then of chloropropanediol). Prior to the atomization, impurities are advantageously removed, in particular using resins and active charcoal.

The invention also relates to the use of atomization for iodinated products, ionic or nonionic iodinated monomers or dimers (or mixtures of ionic or nonionic monomers and/or dimers), for which this process is accompanied by a modification of physicochemical properties of the product (in particular solubility in water) in comparison with the same product crystallized. The products concerned are in particular iopamidol, iohexyl, and iomeprol. The results obtained with ioxilan open the way to the identification of other isolation processes which improve the physicochemical properties of the product, for example by any drying process, freeze drying process or the like.

1. Process for the preparation of the isolated compound ioxilan of general formula (I), characterized in that it comprises the atomization of an aqueous solution of ioxilan:

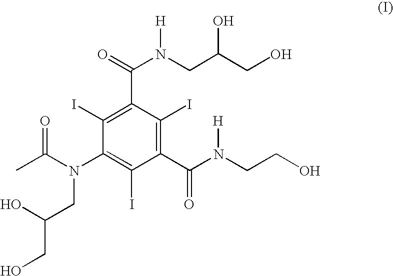

2. Process according to claim 1, wherein the outlet temperature of the atomization device is less than 130° C.

3. Process according to claim 1, wherein the aqueous ioxilan solution results from a process for the manufacture of ioxilan without a crystallization stage.

4. (canceled)

5. Process for the preparation of a pharmaceutical composition of a compound of general formula (I), 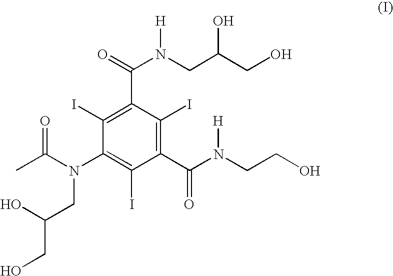
wherein it comprises the aqueous dissolution of the atomized ioxilan (B2) obtained in claim 1 and of pharmaceutically acceptable excipient(s).

6. (canceled)

7. Process according to claim 2 wherein the outlet temperature of the atomization device is of between 100 and 110° C.
